(19) United States
(12) Patent Application Publication
(10) Pub. No.: US 2013/0060107 A1
(43) Pub. Date: Mar. 7, 2013

(54) SUBCUTANEOUS GLUCOSE SENSOR

(71) Applicants: Barry Colin Crane, Shennington (GB); Alasdair Allan Mackenzie, Buckinghamshire (GB); Neil Cairns, Paisley (GB); John Gilchrist, Helensburgh (GB)

(72) Inventors: Barry Colin Crane, Shennington (GB); Alasdair Allan Mackenzie, Buckinghamshire (GB); Neil Cairns, Paisley (GB); John Gilchrist, Helensburgh (GB)

(21) Appl. No.: 13/579,240

(22) PCT Filed: Feb. 15, 2011
(86) PCT No.: PCT/GB11/00208

§ 371 Date: Nov. 21, 2012

Related U.S. Application Data
(60) Provisional application No. 61/306,358, filed on Feb. 19, 2010.

(51) Int. Cl. A61B 5/1459

(52) U.S. Cl. 600/316

(57) ABSTRACT

A glucose sensor for measurement of glucose in subcutaneous tissue, the sensor comprising: a probe for subcutaneous insertion, the probe containing an indicator system comprising a receptor for selectively binding to glucose and a fluorophore associated with said receptor, wherein the fluorophore has a fluorescence lifetime of less than 100 ns; a detector head which is optically connected to the probe and which is for location outside the body; a light source; and a detector arranged to receive fluorescent light emitted from the indicator system, wherein the light source and detector are optionally located within the detector head; wherein the sensor is arranged to measure glucose concentration in subcutaneous tissue by monitoring the fluorescence lifetime of the fluorophore.

### FIELD OF THE INVENTION

The present invention relates to a sensor for measuring glucose in subcutaneous tissue and a method of subcutaneous glucose measurement.

### BACKGROUND TO THE INVENTION

Outcomes Studies on Type 1 and Type 2 diabetes patients (The Diabetes Control and Complications Trial, Epidemiology of Diabetes Interventions and Complications, and United Kingdom Prospective Diabetes Study) have indicated that better control of glucose by frequent monitoring and application of therapies or dietary regimes improves patients outcomes (reduced eye, kidney and nerve desease and a reduced risk of cardiovascular desease and stroke.). However there is a user resistance to frequently sampling blood by finger stick and then measuring the glucose concentration on the many handheld glucometers that are available.

A further difficulty with the currently used glucose monitoring technique is that it provides only intermittent measurement of glucose levels. With “brittle” diabetics the glucose fluctuations are often large and frequent and difficult to bring under control and hence continuous monitoring of glucose is an obvious advantage—particularly during sleep as a guard against hypoglycaemia.

In some cases, ambulatory insulin infusion pumps are implanted into the diabetic patient. In such patients continuous monitoring of glucose is a necessity to avoid inadvertent hypoglycaemia.

Measurement of glucose continuously by the home based diabetic must occur via a practical access site. It would not be feasible for the home diabetic to access a vein or artery to place a sensor. However, subcutaneous tissue has been identified as a viable access point. Continuous glucose sensors that access glucose through subcutaneous tissue have been developed and have usually been based on electrochemical technology and glucose selective enzymes such as glucose oxidase. These sensors are susceptible to denaturing of the enzyme, particularly in a biological environment. Further, because they are consumptive of glucose and rely on constant diffusion of glucose to the sensor electrodes, they are susceptible to errors and drift. For sensors that are “implanted” for 1-5 days or longer, sensor drift is a major issue.

Thus, the currently available technologies present significant barriers to the development of a viable glucose sensor for continuous monitoring of glucose in the home environment.

An alternative technology to the electrochemical devices is the use of optical sensors, such as those based on fluorescence intensity measurements. For instance, reversible, non-consumptive fluorescent optical sensors utilizing fluorophore boronic acid chemistries as the indicator for glucose have been developed. Such sensors measure the change in the emitted fluorescent intensity as a means of determining glucose concentration. Such boronic acid glucose indicating chemistries have the advantage of being reversible with glucose, non-consumptive and are more stable than the enzymes, such as glucose oxidase, which are commonly used on electrochemical glucose sensors. They can also be readily immobilized, within a hydrogel, onto an optical fibre.

A particular disadvantage with such fluorescence intensity measuring devices, however, is the need for calibration of the device. For fluorescence intensity measurements, the emission signal is dependent on the indicator concentration, the path length and the excitation intensity. To provide an accurate reading, calibration of the device is therefore essential. A further difficulty with fluorescence intensity measurement is that the indicators can suffer from photobleaching, which is exhibited as sensor drift, making regular recalibration necessary. User compliance is a particular issue in the consideration of calibration of home use sensors so the need for recalibration, or indeed calibration at all, is undesirable.

Thus, despite the significant work which has gone into the development of suitable glucose sensors for home use, there remains a need for a glucose sensor suitable for continuous monitoring of glucose in the home environment. The sensor should be non-invasive or use a viable access point such as subcutaneous tissue. Furthermore, the sensor should minimise or avoid the difficulties of sensor drift and ideally avoid the need for calibration by the user.

### SUMMARY OF THE INVENTION

The present invention provides a subcutaneous optical sensor, adapted for home use for example by the diabetic patient, which aims to address these difficulties. The sensor of the invention makes use of the change in fluorescence lifetime of a fluorophore and accurately measures glucose concentration in subcutaneous tissue by monitoring the lifetime of a particular type of fluorophore.

The fluorescent lifetime of an indicator is an intrinsic property and is independent of changes in light source intensity, detector sensitivity, light through put of the optical system (such as an optical fibre), immobilized sensing thickness and indicator concentration. In addition, photo bleaching of the fluorophore, that translates to signal drift when fluorescence intensity is measured, is of much smaller significance when fluorescent lifetimes are measured. This means that in contrast to intensity based measurements, no compensation for these variables is required when fluorescent lifetimes are measured. Thus for the end user of such a device this means that there is no need for calibration or recalibration. Lifetime measurement of subcutaneous glucose therefore has significant benefits over intensity based measurement in terms of sensor performance, calibration and ease of use for the end user.

However, there are considerable barriers in the art to the development of practically useful lifetime measuring devices. The instrumentation required for the accurate measurement of fluorescent lifetimes is at present expensive and bulky. This makes it unsuitable for development into a sensor for home use, where small, inexpensive and easy to handle instrumentation is an overriding requirement.

The use of long lifetime (>100 ns) fluorescent metal-ligand/boronic acid complexes as indicators for the optical measurement of glucose can facilitate the use of small, low cost instrumentation, such as a light emitting diode for excitation, a photodiode detector, phase fluorimetry and a look up table. There is a problem, however, in using such long lifetime fluorophores for measuring glucose. Long lifetime fluorophores invariably undergo collisional fluorescence quenching with oxygen and the extent of the quenching is proportional to the unquenched lifetimes. Metal ligand complexes with long fluorescent lifetimes are commonly used for the detection and determination of oxygen. Thus oxygen can be regarded as an intereferent when these long lifetime indicators are used for monitoring glucose in tissue, interstitial fluid or blood or some other body fluid. Oxygen interference is a particular problem with subcutaneous glucose measurement in diabetics, where oxygen transport to the peripheral tissues may be compromised and variable, and the sensor is typically located very near to the tissue surface.

The sensor of the invention, however, addresses these issues by providing particular devices capable of measuring lifetimes of less than 100 ns using small, low cost instrumentation. The present invention thus enables the benefits of lifetime measurement to be achieved in a sensor appropriate for home use, and eliminates or reduces the difficulties of oxygen sensitivity.

The present invention therefore provides a glucose sensor for measurement of glucose in subcutaneous tissue, the sensor comprising:
a probe for subcutaneous insertion, the probe containing an indicator system comprising a receptor for selectively binding to glucose and a fluorophore associated with said receptor, wherein the fluorophore has a fluorescence lifetime of less than 100 ns;
a detector head which is optically connected to the probe and which is for location outside the body;
a light source; and
a detector arranged to receive fluorescent light emitted from the indicator system, wherein the light source and detector are optionally located within the detector head;
   wherein the sensor is arranged to measure glucose concentration in subcutaneous tissue by monitoring the fluorescence lifetime of the fluorophore.

According to a preferred embodiment, the detector is a single photon avalanche diode. The intensity of light emitted by the light source is modulated at a first frequency, and the bias voltage applied to the single photon avalanche diode is modulated at a second frequency, different from the first frequency. The bias voltage is above the breakdown voltage of the single photon avalanche diode. This selection of bias voltage means that the single photon sensitivity of the detector is maintained, but also has the advantage that a heterodyne measurement approach can be used. In other words, the resulting measurement signal of interest from the single photon avalanche diode is at a frequency corresponding to the difference between the first and second frequencies. The first and second frequencies may be of the order of 1 MHz or much higher, but may be selected such that their difference is, for example, of the order of 10 s of kHz. Therefore, the operational bandwidth of the measurement electronics can be much lower than the first and second modulation frequencies, allowing a simpler design and with less sensitivity to noise.

A further advantageous aspect is to introduce a series of additional phase angles (or time delays equivalent to phase shifts) in the modulation signal for the light source. A series of measurements can then be obtained relating the modulation depth of the measurement signal to the introduced phase angle. Analysing these results can improve the overall precision of the fluorescence lifetime measurement.

Also provided is a disposable probe unit for use in a glucose sensor of the invention, comprising (a) a probe for subcutaneous insertion, the probe containing an indicator system of the invention, and (b) a connector arranged to optically connect the probe to a detector head comprising, or being itself further optically connected to, a light source and a detector.

Also provided is a detector head adapted for connection to a separate probe unit, wherein the detector head comprises a detector which is a single photon avalanche diode, the detector being arranged to receive light from the probe unit, the detector head being adapted to monitor fluorescence lifetimes of less than 100 ns.

Also provided is a method of measuring glucose concentration in subcutaneous tissue which comprises

(a) inserting the probe of a sensor of the invention into subcutaneous tissue;
(b) providing incident light to the indicator system from the light source;
(c) receiving fluorescent light, emitted from the indicator system in response to the light incident on the indicator system from the light source, using the detector and generating an output signal; and
(d) determining information related to the fluorescence lifetime of the fluorophore based on at least the output signal of the detector.

### BRIEF DESCRIPTION OF THE FIGURES

FIG. 1 depicts a subcutaneous glucose sensor of the invention;

FIG. 2 depicts separately the probe and detector head which make up the sensor of the invention as well as the reader unit.

FIG. 3 schematically shows electronic apparatus contained in the detector head and in the reader unit in one embodiment of the invention.

FIG. 4 depicts the apparatus of a sensor according to a preferred embodiment.

FIG. 5 is a flowchart of a glucose concentration measurement method according to a preferred embodiment of the invention

### DETAILED DESCRIPTION OF THE INVENTION

As used herein the term alkylene is a linear or branched alkyl moiety containing, for example, from 1 to 15 carbon atoms such as a C₁₋₁₂ alkylene moiety, C₁₋₆ alkylene moiety or a C₁₋₄ alkylene moiety, e.g. methylene, ethylene, n-propylene, i-propylene, n-butylene, i-butylene and t-butylene. For the avoidance of doubt, where two alkylene moieties are present in a group, the alkylene moieties may be the same or different.

An alkylene moiety may be unsubstituted or substituted, for example it may carry one, two or three substituents selected from halogen, hydroxyl, amine, (C₁₋₄ alkyl) amine, di(C₁₋₄ alkyl) amine and C₁₋₄ alkoxy. Preferably an alkylene moiety is unsubstituted.

As used herein the term aryl or arylene refers to C₆₋₁₄ aryl groups or moieties which may be mono- or polycyclic, such as phenyl, naphthyl and fluorenyl, preferably phenyl. An aryl group may be unsubstituted or substituted at any position. Typically, it carries 0, 1, 2 or 3 substituents. Preferred substituents on an aryl group include halogen, C₁₋₁₅ alkyl, C₂₋₁₅ alkenyl, —C(O)R wherein R is hydrogen or C₁₋₁₅ alkyl, —CO₂R wherein R is hydrogen or C₁₋₁₅ alkyl, hydroxy, C₁₋₁₅ alkoxy, and wherein the substituents are themselves unsubstituted.

As used herein, a heteroaryl group is typically a 5- to 14-membered aromatic ring, such as a 5- to 10-membered ring, more preferably a 5- or 6-membered ring, containing at least one heteroatom, for example 1, 2 or 3 heteroatoms, selected from O, S and N. Examples include thiophenyl, furanyl, pyrrolyl and pyridyl. A heteroaryl group may be unsubstituted or substituted at any position. Unless otherwise stated, it carries 0, 1, 2 or 3 substituents. Preferred substituents on a heteroaryl group include those listed above in relation to aryl groups.

The present invention provides a sensor and measurement technique for the measurement of glucose concentration in subcutaneous tissue. The probe containing the indicator system is inserted into the subcutaneous tissue under the skin. One or more apertures are provided to enable glucose in the surrounding tissue to enter the probe and to bind with the receptor contained in the indicator system. Typically, the probe is in contact with the subcutaneous tissue and interstitial fluid beneath the skin. Glucose from the interstitial fluid therefore enters the probe and the sensor accordingly reflects the concentration of glucose in this interstitial fluid.

The indicator system is contained within the probe and is therefore located under the skin during use of the sensor. The glucose entering the probe therefore quickly contacts the indicator system. The present invention accordingly avoids the time delay associated with devices which transport the glucose to an ex vivo part of the sensor device prior to contact with the indicator.

On contact of the glucose with the indicator system, binding occurs between the receptor and glucose molecules. The presence of a glucose molecule bound to the receptor causes a change in the fluorescence lifetime of the indicator system. Thus, monitoring of the lifetime of the fluorophore in the indicator system provides an indication of the amount of glucose which is bound to the receptor. The measurement of glucose concentration by monitoring the lifetime decay has previously been described by Lakowicz in Analytical Biochemistry 294, 154-160 (2001). Measurement by phase modulation is described therein but both phase modulation and single photon counting techniques are appropriate for use with the present invention. Phase modulation is preferred.

The indicator system contains at least a receptor that selectively binds to glucose and a fluorophore associated with the receptor. The lifetime of the fluorescence decay of the fluorophore is altered when glucose is bound to the receptor, allowing detection of glucose by monitoring the lifetime of the fluorophore. In one embodiment, the receptor and fluorophore are covalently bound to one another.

Suitable receptors for glucose are enzymes and compounds containing one or more, preferably two, boronic acid groups. In a particular embodiment, the receptor is a group of formula (I)

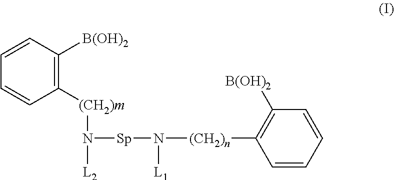

wherein m and n are the same or different and are typically one or two, preferably one; Sp is an alphatic spacer, typically an alkylene moiety, for example a C1-C12 alkylene moiety, e.g. a C6 alkylene moiety; and L1 and L2 represent possible points of attachment to other moieties, for example to a fluorophore. For example, L1 and L2 may represent an alkylene, alkylene-arylene or alkylene-arylene-alkylene moiety, linked to a functional group. Where no attachment to another moiety is envisaged, the functional group is protected or replaced by a hydrogen atom. Typical alkylene groups for L1 and L2 are C1-C4 alkylene groups, e.g. methylene and ethylene, especially methylene. Typical arylene groups are phenylene groups. The functional group is typically any group which can react to form a bond with, for example, the fluorophore or a hydrogel, e.g. ester, amide, aldehyde or azide. In the indicator system, the receptor is typically linked via one or more of these functional groups to the fluorophore and optionally to a support structure such as a hydrogel.

Varying the length of the spacer Sp alters the selectivity of the receptor. Typically, a C6-alkylene chain provides a receptor which has good selectivity for glucose.

Further details of such receptors are found in U.S. Pat. No. 6,387,672, the contents of which are incorporated herein by reference in their entirety.

Receptors of formula (I) can be prepared by known techniques. Further details can be found in U.S. Pat. No. 6,387,672.

It is to be understood that the present invention is not limited to the particular receptors described above and other receptors, particularly those having two boronic acid groups, may also be used in the present invention.

Examples of suitable fluorophores include anthracene, pyrene and derivatives thereof, for example the derivatives described in GB 0906318.1, the contents of which are incorporated herein by reference in their entirety. The fluorophore is typically non-metallic. The lifetime of the fluorophore is typically 100 ns or less, for example 30 ns or less. The lifetime may be 1 ns or more, for example 10 ns or more. Particular examples of suitable fluorophores are derivatives of anthracene and pyrene with typical lifetimes of 1 to 10 ns and derivatives of acridones and quinacridones with typical lifetimes of 10 to 30 ns.

The receptor and fluorophore are typically bound to one another to form a receptor-fluorophore construct, for example as described in U.S. Pat. No. 6,387,672. This construct may further be bound to a support structure such as a polymeric matrix, or it may be physically entrapped within the probe, for example entrapped within a polymeric matrix or by a glucose-permeable membrane. A hydrogel (a highly hydrophilic cross-linked polymeric matrix such as a cross-linked polyacrylamide) is an example of a suitable polymeric matrix. In a preferred embodiment, a receptor-fluorophore construct is covalently bound to a hydrogel, for example via a functional group on the receptor. Thus, the indicator is in the form of a fluorophore-receptor-hydrogel complex.

In an alternative preferred embodiment, the indicator (comprising receptor and fluorophore, typically in the from of a receptor-fluorophore construct) is provided in soluble form, typically, the indicator system is provided as an aqueous solution. This has the particular advantage that the microenvironment surrounding each indicator moiety remains substantially constant. Fluorescent sensors can be dramatically influenced by the microenvironment of the indicator. Variation in the localised microenvironment surrounding the indicator can lead to variation in the fluorescent response. In the case of an indicator immobilised onto a polymeric matrix, there is significant variation in the microenvironment, which can lead to a lifetime decay signal in the form of a continuous distribution of decay times and complex multi exponentials. In contrast, where the indicator is dissolved in a solvent, such as water, particularly at low concentrations such that the indicator molecules do not aggregate and are monodispersed, homogeneity is maximum and ideal fluorescent characteristics are achieved for that given solvent. This leads to a signal which is a simple, single exponential.

An alternative means to achieve homogeneity is to immobilise the indicator onto a single molecule support of large molecular weight. Preferably the support is symmetrical and the spatial attachment of the fluorescent indicator is achieved in such a way that the result is also symmetrical. This can, for example, be achieved by the use of a dendrimer as the support material, as discussed below. Thus the environments of each fluorescent indicator molecule attached to such a support will be equivalent. In addition if such a supported molecule can be dissolved in a solvent, such as water, at an appropriate concentration, the environments of the supported indicator will be homogenous, again leading to improved signal characteristics.

In this embodiment, therefore, the indicator (e.g. receptor-fluorophore construct) may be contained within the probe in aqueous solution and a membrane, which is permeable to glucose, provided over any aperture in the probe. The membrane restricts the passage of the indicator in order to ensure that the indicator remains within the cell. This is typically achieved by ensuring that the indicator is of sufficiently high molecular weight to be substantially prevented from passing through the membrane, and by use of a membrane having a suitable molecular weight cut-off. Dialysis membranes are appropriate for use in the present invention.

In some instances, the indicator may inherently be of sufficiently high molecular weight to prevent its passage through the membrane. As discussed above, this provides maximum homogeneity in the microenvironment surrounding the indicator. In this instance, the indicator system may be in the form of a solution of the indicator. Alternatively, the receptor and fluorophore may be bonded to a support material to provide a complex of support, receptor and fluorophore, the complex being dissolved in the solution. The nature of the complex is not important as long as the receptor and fluorophore remain bonded to the support. For example, the support material may be bonded to a receptor-fluorophore construct. Alternatively, the support material may be bonded separately to the fluorophore and to the receptor. In the latter case, the receptor and fluorophore are not directly bonded to one another but are linked only via the support material. In one embodiment of the invention, the complex takes the form fluorophore-receptor-support.

Typically, a high molecular weight support material is used. This enables the skilled person to restrict the passage of the indicator through the membrane by providing the indicator within a higher molecular weight complex. Preferred support materials have a molecular weight of at least 500, for example at least 1000, 1500 or 2000 or 10,000. The support material should also be soluble in water, and should be inert in the sense that it does not interfere with the sensor itself.

Suitable materials for use as the support material include polymers. Any non-cross-linked, linear polymer which is soluble in the solvent used can be employed. Alternatively, the support material may be a cross linked polymer (e.g. a lightly cross-linked polymer) that is capable of forming a hydrogel in water. For example, the support material may be a hydrogel formed from a cross-linked polymer having a water content of at least 30% such that there is no distinct interface between the polymer and aqueous domains.

Polyacrylamide and polyvinylalcohol are examples of appropriate water-soluble, linear polymers. Preferably, the polymer used has a low polydispersity. More preferably, the polymers are uniform (or monodisperse) polymers. Such polymers are composed of molecules having a uniform molecular mass and constitution. The lower polydispersity leads to an improved sensor modulation. Cross-linked polymers for formation of hydrogels may be formed from the above water-soluble linear polymers cross-linked with ethylene glycol dimethacrylate and/or hydroxylethyldimethacrylate.

In one embodiment, the indicator is bound to a hydrogel having a high water content. In this instance, the indicator system typically comprises an aqueous solution containing the hydrogel. The water content of the hydrogel is so high, preferably at least 30% w/w, that the solution/hydrogel mixture can be considered a mixture of fluids with no distinct solid interfaces between the polymer and aqueous domains. As used herein, a fluid hydrogel is a hydrogel having a water content which is so high (typically at least 30% w/w) that there are no distinct solid interfaces between the polymer and aqueous domains when the hydrogel is placed in water. Such a hydrogel may comprise a lightly cross-linked polymer which may dissolve in the solvent, or which may form a fluid hydrogel with a relatively low water content; alternatively, the hydrogel may comprise a more heavily cross-linked polymer having a higher water content such that it is in the form of a fluid.

In a particularly preferred aspect, the support material is a dendrimer. The nature of the dendrimer for use in the invention is not particularly limited and a number of commercially available dendrimers can be used, for example polyamidoamine (PAMAM), e.g. STARBURST® dendrimers and polypropyleneimine (PPI), e.g. ASTRAMOL® dendrimers. Other types of dendrimers that are envisaged include phenylacetylene dendrimers, Frechet (i.e. poly(benzylether)) dendrimers, hyperbranched dendrimers and polylysine dendrimers. In one aspect of the invention a polyamidoamine (PAMAM) dendrimer is used.

Dendrimers include both metal-cored and organic-cored types, both of which can be employed in the present invention. Organic-cored dendrimers are generally preferred.

The properties of a dendrimer are influenced by its surface groups. In the present invention, the surface groups act as the binding point for attachment to the receptor and the fluorophore. Preferred surface groups therefore include functional groups which can be used in such binding reactions, for example amine groups, ester groups or hydroxyl groups, with amine groups being preferred. The nature of the surface group, however, is not particularly limited. Some conventional surface groups which could be envisaged for use in the present invention include amidoethanol, amidoethylethanolamine, hexylamide, sodium carboxylate, succinamic acid, trimethoxysilyl, tris(hydroxymethyl)amidomethane and carboxymethoxypyrrolidinone, in particular amidoethanol, amidoethylethanolamine and sodium carboxylate.

The number of surface groups on the dendrimer is influenced by the generation of the dendrimer. Preferably, the dendrimer has at least 4, more preferably at least 8 or at least 16 surface groups. Typically, all of the surface groups of the dendrimer will be bound to a receptor or fluorophore moiety. However, where some surface groups of the dendrimer remain unbound to a receptor or fluorophore moiety (or a construct of receptor and fluorophore), the surface groups may be used to impart particular desired properties. For example, surface groups which enhance water-solubility such as hydroxyl, carboxylate, sulphate, phosphonate or polyhydroxyl groups may be present. Sulphate, phosphonate and polyhydroxyl groups are preferred examples of water soluble surface groups.

In one aspect, the dendrimer incorporates at least one surface group which contains a polymerisable group. The polymerisable group may be any group capable of undergoing a polymerisation reaction, but is typically a carbon carbon double bond. Examples of suitable surface groups incorporating polymerisable groups are amido ethanol groups wherein the nitrogen atom is substituted with a group of formula -linker-C═CH₂. The linker group is typically an alkylene, alkylene-arylene, or alkylene-arylene-alkylene group wherein the alkylene is typically a C1 or C2 alkylene group and arylene is typically phenylene. For example, the surface group may comprise an amidoethanol wherein the nitrogen atom is substituted with a —CH₂—Ph—CH═CH₂ group.

The presence of a polymerisable group on the surface of the dendrimer enables the dendrimer to be attached to a polymer by polymerising the dendrimer with one or more monomers or polymers. Thus, the dendrimer can be tethered to, for example, a water soluble polymer in order to enhance water solubility of the dendrimer, or to a hydrogel (i.e. a highly hydrophilic cross-linked polymer matrix, e.g. of polyacrylamide) to assist in containing the dendrimer within the cell.

Preferably the dendrimer is symmetrical, i.e. all of the dendrons are identical.

The dendrimer may have the general formula:

CORE−[A]ₙ

wherein CORE represents the metal or organic (preferably organic) core of the dendrimer and n is typically 4 or more, for example 8 or more, preferably 16 or more. Examples of suitable CORE groups include benzene rings and groups of formula —RN—(CH₂)ₚ—NR— and >N—(CH₂)ₚ—N< where p is from 2 to 4, e.g. 2 and R is hydrogen or a C1-C4 alkyl group, preferably hydrogen. —HN—(CH₂)₂—NH— and >N—(CH₂)₂—N< are preferred.

Each group A may be attached either to the CORE or to a further group A, thus forming the typical cascading structure of a dendrimer. In a preferred aspect, 2 or more, for example 4 or more, groups A are attached to the CORE (first generation groups A). The dendrimer is typically symmetrical, i.e. the CORE carries 2 or more, preferably 4 or more, identical dendrons.

Each group A is made up of a basic structure having one or more branching groups. The basic structure typically comprises alkylene or arylene moieties or a combination thereof. Preferably the basic structure is an alkylene moiety. Suitable alkylene moieties are C1 -C6 alkylene moieties. Suitable arylene moieties are phenylene moieties. The alkylene and arylene moieties may be unsubstituted or substituted, preferably unsubstituted, and the alkylene moiety may be interrupted or terminated with a functional group selected from —NR′—, —O—, —CO—, —COO—, —CONR′—, —OCO—and —OCONR′, wherein R′ is hydrogen or a C1-C4 alkyl group.

The branching groups are at least trivalent groups which are bonded to the basic structure and have two or more further points of attachment. Preferred branching groups include branched alkyl groups, nitrogen atoms and aryl or heteroaryl groups.

Nitrogen atoms are preferred.

The branching groups are typically bonded to (i) the basic structure of the group A and (ii) to two or more further groups A. Where on the surface of the dendrimer, however, the branching group may itself terminate the dendrimer (i.e. the branching group is the surface group), or the branching group may be bonded to two or more surface groups.

Examples of preferred groups A are groups of formula

—(CH₂)_{q}—(FG)ₛ—(CH₂)ᵣ—H₂

wherein q and r are the same or different and represent an integer of from 1 to 4, preferably 1 or 2, more preferably 2. s is 0 or 1. FG represents a functional group selected from —NR′—, —O—, —CO—, —COO—, —CONR′—, —OCO— and —OCONR′, wherein R′ is hydrogen or a C1-C4 alkyl group. Preferred functional groups are —CONH—, —OCO— and —COO—, preferably —CONH—.

A discussed above, the surface group forms the point of attachment of the dendrimer to the indicator (or separately to the receptor and fluorophore moieties). The surface groups therefore typically include an unsubstituted or substituted alkylene or arylene moiety or a combination thereof, preferably an unsubstituted or substituted alkylene moiety, and at least one functional group which is suitable for bonding to the indicator. The functional group is typically an amine or hydroxyl group, with amine groups being preferred. Particular examples of surface groups are provided above.

An example of a dendrimer which can be employed in the present invention is a PAMAM dendrimer of generation 1 or 2 synthsised in accordance with Cheng et al (European Journal of Medicinal Chemistry, 2005, 40, 1384-1389). The resulting surface amine groups can be used to bind to suitable receptor or fluorophore moieties, or receptor-fluorophore constructs.

Where the dendrimer employed is a metal-cored dendrimer, it may itself have fluorescent properties. In this case, it is envisaged that the dendrimer itself may form the fluorophore moiety. The support-bound indicator in this case simply comprises a receptor moiety bound to the dendrimer.

In a further aspect, the support material is a non-dendritic, non-polymeric macromolecule having high molecular weight (i.e. at least 500, preferably at least 1000, 1500 or 2000 or 10,000). Cyclodextrins, cryptans and crown ethers are examples of such macromolecules. Such macromolecules also provide a uniform environment for the indicator and lead to a more consistent fluorophore response to analyte binding.

The receptor and fluorophore may be bonded to the support material by any appropriate means. Covalent linkages are preferred. Typically, the fluorophore and receptor are linked to form a fluorophore-receptor construct, which is then bound to the support material. Alternatively, the receptor and fluorophore may be separately bound to the support material. The number of receptor-fluorophore construct moieties per support material moiety is typically greater than 1, for example 4 or more, or 8 or more. Where a dendritic support material is used, the surface of the dendrimer may be covered with indicator moieties. This may be achieved by binding an indicator moiety to all (or substantially all) of the surface dendrons.

Where a polymeric support material is used, the receptor-fluorophore construct may be modified to include a double bond and copolymerised with a (meth)acrylate or other appropriate monomer to provide a polymer bound to the indicator. Alternative polymerisation reactions, or simple addition reactions, may also be employed. Wang et al (Wang B., Wang W., Gao S., (2001), Bioorganic Chemistry, 29, 308-320) provides an example of a polymerisation reaction including a monoboronic acid glucose receptor linked to an anthracene fluorophore.

In the case of a dendritic support material, the dendrimer is either reacted separately with the fluorophore and receptor moieties, or more preferably is reacted with a pre-formed receptor-fluorophore construct. Any appropriate binding reaction may be used. An example of a suitable technique is to react a dendrimer having surface amine groups with a fluorophore-receptor construct having a reactive aldehyde group by reductive amination in the presence of a borohydride type reagent. The resulting structure can be purified by ultrafiltration. An example of a dendrimer bound to a boronic acid receptor and an anthracene fluorophore is provided by James et al (Chem. Commum., 1996 p 706).

In the case of the dendritic support material having a polymerisable group as a surface group, the dendrimer may undergo a polymerisation reaction with one or more monomers in order to form a dendrimer-polymer construct wherein a polymer is bound to the surface of the dendrimer. Typically, the dendrimer is added at a late stage in the polymerisation reaction so that the dendrimer terminates the polymer chain.

Alternatively, the dendrimer may be reacted with a pre-formed polymer. This can be achieved, for example, by a condensation reaction between a carboxylic acid group on the polymer with a hydroxyl group on the dendrimer, to provide the link through the formed ester.

Examples of monomers and polymers which can be used in these reactions are (meth)acrylate, (meth)acrylamide and vinylpyrrolidone and combinations thereof and their corresponding polymers. Preferred polymers are water soluble polymers. Preferably, the water-solubility of the polymer is such that adequate fluorescent signal is produced when the polymer/indicator is dissolved in water (ideally infinite solubility). Polyacrylamide is particularly preferred since this leads to the formation of a highly water soluble polyacrylamide chain attached to the dendrimer. In one aspect of this embodiment, the polymer (e.g. polyacrylamide) chain bound to the dendritic support material is cross-linked to form a hydrogel. Optionally, the hydrogel has a high water content such that when placed in water there is no distinct interface between the aqueous phase and the polymer phase (as used herein, the hydrogel is in fluid form). In this case, it is typically provided in the form of a mixture with water or an aqueous solution.

Polymerisation from the surface of the dendrimer may be carried out either before or after attachment of the fluorophore and receptor moieties.

In the case of a the receptor and fluorophore being provided to the sensor in aqueous solution, a suitable concentration of receptor-fluorophore construct or support bound construct is 10⁻⁶ to 10⁻³M . The concentration may be varied dependent on the required sensor properties. The higher the concentration or amount of receptor and fluorophore in the solution, the greater the signal level.

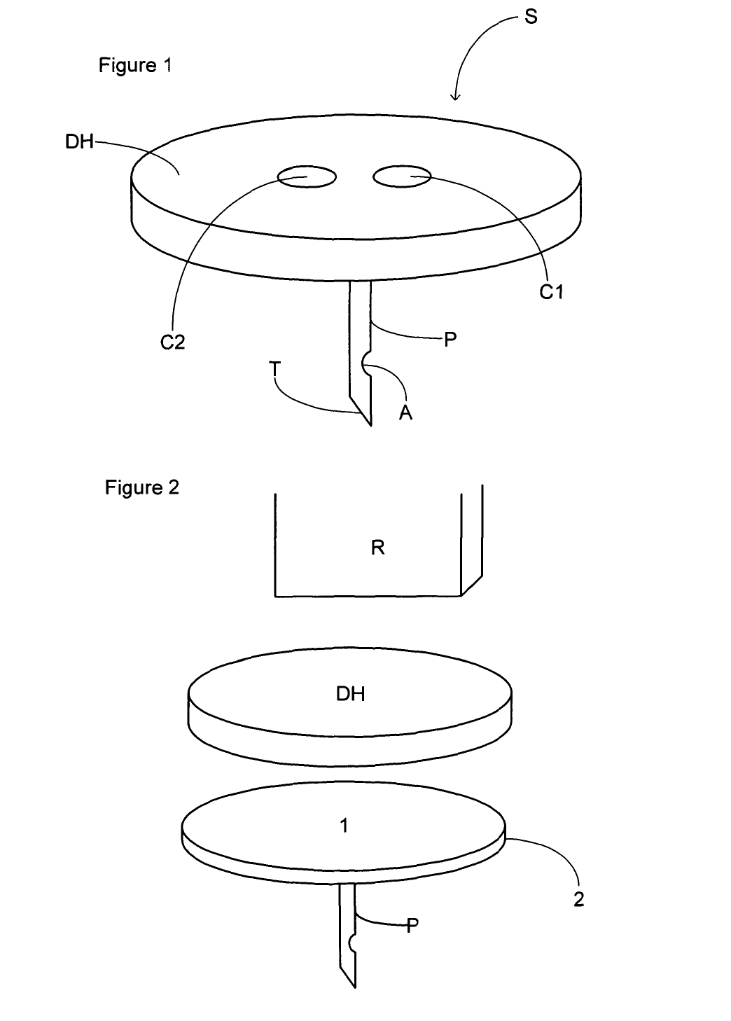

One embodiment of a sensor of the invention is depicted in FIGS. 1 and 2. FIG. 1 shows a sensor unit S which comprises two parts: a detector head DH that provides an ex vivo base on which to locate a probe and may contain a memory device, any necessary optics and electronics, a battery or other power source and optionally the light source and detector; and a probe P that contains the indicating chemistry and waveguide. The detector head is typically at least 2 mm in thickness (e.g. 2-5 mm) and has a diameter of approximately lcm (e.g. 0.5-3 cm). FIGS. 1 and 2 depict disc-shaped detector heads, but the shape of the detector head can be varied.

A probe P is also provided which is inserted into the body during use. The probe typically has a tapered tip T to facilitate insertion into the skin and to minimise tissue damage during insertion. The probe is typically cylindrical in shape and preferably has a length of at least 3 mm, for example up to 12 mm. The diameter of the probe is typically no more than 0.5 mm, for example from 0.1 mm to 0.5 mm. An example of a suitable probe is a cylindrical hollow needle (optionally with the end capped to prevent entry of body fluids or tissue). The probe thus has a length which is suitable for probing interstitial tissue and is generally shorter than a corresponding probe used for an intravascular measurement. A probe for intravascular use typically has a length of at least several cm and normally will be significantly longer and suitable for insertion into a blood vessel via a cannula.

The indicator system is contained within the probe. Glucose is able to enter the probe from the interstitial fluid via aperture A so that binding with the receptor can occur. As here depicted, a single aperture A is provided in the longitudinal wall of the probe. Two or more apertures may be present if desired. Such apertures in the longitudinal wall of the probe are preferably close to the tip of the probe. Alternatively or additionally, an aperture may be provided in the tip of the probe.

The probe is typically designed such that the distance from the top of the probe (where the probe meets the detector head or the connector) to the (or each) aperture A is no more than 10 mm, preferably no more than 8 mm or 5 mm. When a sensor having such a probe is inserted into the skin such that the detector head or connector rests against the skin, the (or each) aperture A is located subcutaneously, such that in interstitial fluid is able to enter the probe through the (or each) aperture A.

The indicator system is typically fixed within the probe at or close to aperture A in order to ensure rapid diffusion of glucose to the indicator. In one embodiment, the receptor/fluorophore are provided in a hydrogel or other polymeric matrix and the hydrogel is located within the hollow bore of the probe, or within a hole in the probe provided for such use. Alternatively, the indicator may be provided in aqueous solution within a cell within probe P. Glucose-permeable membrane is preferably placed across the aperture A to maintain the indicator system within the probe and allow entry of glucose.

In one embodiment of the invention, the fluorescent signal may be temperature corrected. In this embodiment, a thermocouple (thermistor or other temperature probe) will be placed beside the indicating chemistry in the probe.

As depicted in FIG. 2, the sensor unit may be provided in two separable parts. A first part is the probe unit 1 which comprises the probe P and optionally a connector 2 for connecting the probe to the detector head. The second part is the detector head DH. The connector is arranged to optically connect the waveguide in the probe to the detector head DH in use, such that optical connection between the indicator system and the light source and detector is maintained. Typically, a bifurcated waveguide will be provided in the detector head, one side interfacing with the light source and the other with the detector. In the case that a thermocouple is provided in the probe, a further connection is provided to the thermocouple. The detector head and probe will also typically have a locking mechanism in order to correctly align any connections. Once connected, the probe, connector and detector head make up the sensor unit of FIG. 1.

In this embodiment, it is envisaged that the probe unit 1 will be a disposable unit having a connector made of a low cost material such as a synthetic polymer. The probe may be a needle such as a stainless steel or titanium needle. The detector head DH is in this embodiment a non-disposable unit which is arranged to connect to a new probe unit for each use. A power source, for example a rechargeable battery or unit arranged to contain disposable battery, may also be located within the detector head.

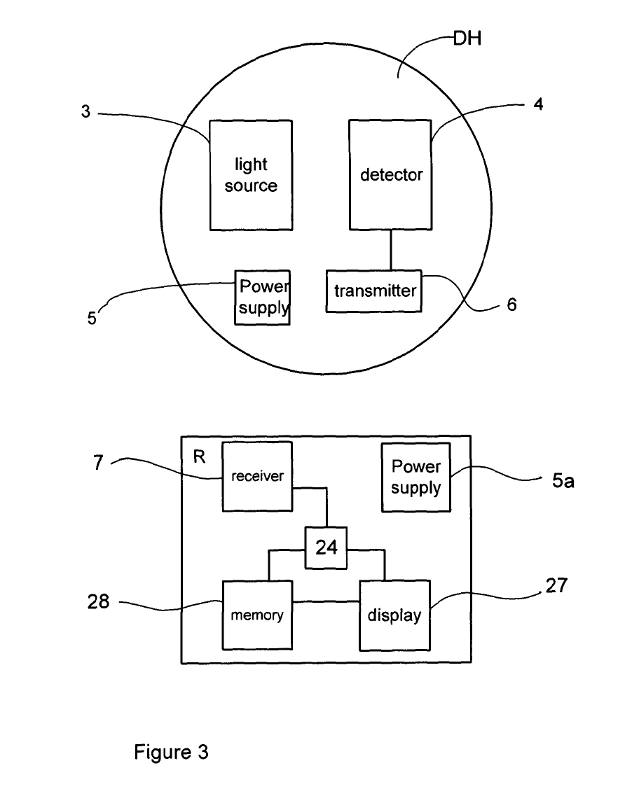

The sensor unit is used in conjunction with a reader unit R, a preferred embodiment of which is depicted in FIG. 3. The reader unit typically provides an output of the glucose concentration which can displayed on a display 27 or stored in a memory 28. The reader unit additionally contains any necessary power supply 5a (e.g. rechargeable battery or unit arranged to contain disposable batteries), processing unit 24 and other necessary electronics. The reader unit may have a connector for physically connecting to the detector head to provide either electronic, or electronic and optical, connection. For example, connection may be made through contact between touch contacts C1 and C2 on the top of the detector head with similar touch contacts on the reader unit (not depicted) or via cable connection. The reader unit may be physically clipped into the detector head during use. Alternatively, the reader unit may be arranged to receive data from the detector head other than via physical connection, for example via induction or through wireless transmission of data. In this case, the reader unit contains a receiver arranged to receive data transmitted from the detector head. Wireless transmission or connection via touch contacts is preferred.

Also provided in the sensor of the invention is a light source 3 for transmitting incident light of appropriate wavelength to the indicator and a detector 4 for detecting a return signal. As depicted in FIG. 3, these are typically contained in the detector head. The light source is preferably an LED but may be an alternative light source such as a laser diode. The light source may be temperature stabilised. The wavelength of the light source will depend on the fluorophore used. The term “light” is not intended to imply any particular restriction on the emission wavelength of the light source, and in particular is not limited to visible light. The light source 3 may include an optical filter to select a wavelength of excitation, but this filtering may be unnecessary if the light source has a sufficiently narrow band or is monochromatic.

Any appropriate detector 4 capable of detecting fluorescence lifetimes may be used. In one aspect the detector 4 is a single photon avalanche diode (SPAD) (a type of photodiode); suitable SPADs include SensL SPMMicro, Hamamatsu MPPC, Idquantique ID101, and other similar devices. (A single-photon avalanche diode may also be known as a Geiger-mode APD or G-APD; where APD stands for avalanche photodiode.) An optical filter (not shown) may be provided to restrict the wavelengths of light that can reach the detector 4, for instance to block substantially all light except that at the fluorescence wavelength of interest.

A waveguide is typically provided to transmit light between the light source/detector and the indicator system. Where the detector and light source are located close to the end of the probe, a waveguide may be dispensed with (or the probe itself may act as a waveguide). Alternatively, a waveguide such as an optical fibre may be used. If desired, the indicator system may be attached to the tip of the optical fibre, or within the distal end of the fibre and the fibre inserted into the probe such that the indicator system is located at or close to aperture A.

In the depicted embodiment, the light source and detector are present in the detector head. This has the advantage that no optical connection is required between the sensor head and the reader unit. In an alternative embodiment, the light source and detector are located within the reader unit. This has the advantage that a simple, small and light detector head may be used, since this part may, for example, contain only a memory device and any necessary optics. However, a reliable optical connection must be established between the reader unit and the detector head. This can be achieved by use of an optical cable connecting the reader unit and detector head.

In one embodiment of the invention, depicted in FIG. 3, the detector head additionally comprises a power supply 5. The power supply may be a rechargeable battery unit or a unit arranged to contain disposable batteries. This embodiment has the advantage that measurement of glucose concentration in subcutaneous tissue can be carried out without physically connecting the sensor unit and the reader unit. This embodiment is therefore particularly useful in continuous glucose monitoring, for example monitoring glucose levels overnight. The detector head may contain a small memory capacity to store the obtained data.

In a preferred aspect of this embodiment, the detector head further comprises a transmitter 6. In this embodiment, the lifetime data collected by the sensor unit can be transmitted wirelessly to a receiver 7 located in the reader unit. Typically, the output signal from the detector is transmitted, optionally after conversion to a digital signal (e.g. via suitable analogue-to-digital converter (ADC), not depicted). Such transmission may be carried out, for example, by induction, by infra-red or by other suitable means for wireless transmission of data such as via wireless telephone or internet connection. In this way, the reader unit and sensor unit may be distant from one another. For example, the reader unit may be at a fixed location within the patient's home and the patient can freely move about the home or the locality whilst data continues to be collected and transmitted to the reader. Similarly, the reader unit may be provided in a hospital whilst the sensor unit is fixed to the patient at home. An example of the transmission of medical data in this manner can be found in WO 99 59460. The systems for transmission and receipt of data described in that application can be employed in the present invention.

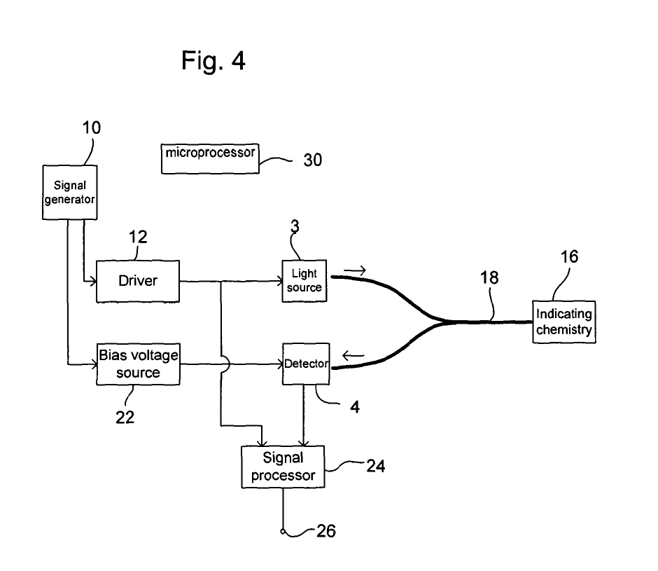

FIG. 4 shows schematically a preferred embodiment of a fluorescence sensor according to the invention which uses a SPAD detector. This embodiment describes the measurement of the lifetime of the fluorophore using frequency domain measurements, but the same apparatus can equally be used for time domain measurements. A signal generator 10 produces a high frequency periodic signal at a first frequency that is passed to a driver 12. The driver 12 may condition the first signal and then uses it to drive modulation of the light source 3. Typically, the signal generator and driver are contained in the detector head together with the light source and detector, although in alternative embodiments they may be present in the reader unit.

The driver 12 drives the light source 3 to modulate the intensity (amplitude) of the excitation light. Preferably this is done by the driver 12 electrically modulating the light source to vary the emission intensity. Alternatively, the light source 3 may include a variable optical modulator to change the final output intensity. The shape (waveform) of the modulation of the intensity of the light from the light source 3, controlled by the signal generator 10 and the driver 12, may take various forms depending on the circumstances, including sinusoidal, triangular or pulsed, but the modulation is periodic at the first frequency.

The light output from the light source 3 is transmitted to the indicating chemistry 16 within the probe, in FIG. 4 via an optical fibre 18, although alternative waveguide, e.g. the probe alone, may be used. In this embodiment, because the output of the light source 3 is periodically modulated, then the fluorescence light is also modulated in nature at the same fundamental first frequency. However, there is a time delay introduced in the fluorescence emitted light because of the fluorescence behaviour of the fluorophore; this manifests itself as a phase delay between the modulation of the excitation light and the modulation of the fluorescence light.

The emitted fluorescence light is transmitted to a detector 4 via optical fibre 18. In this embodiment, detector 4 is a single photon avalanche diode (SPAD). The single photon avalanche diode detector 4 can be either the kind having a low breakdown voltage (threshold) or a high breakdown voltage. A bias voltage may be applied to the single photon avalanche diode detector by a bias voltage source 22, such that the bias voltage is above the breakdown voltage of the single photon avalanche diode. In this state the detector 4 has very high sensitivity such that receipt of a single photon causes an output current pulse, and thus the total output current is related to the received light intensity, even when the intensity is very low.

The bias voltage source 22 receives a periodic signal at a second frequency from the signal generator 10 such that the bias voltage applied to the single photon avalanche diode detector 4 is modulated at that second frequency. In the preferred embodiment, the single photon avalanche diode detector is a low voltage type and the mean bias voltage is in the region of 25 to 35 Vdc, but may be higher or lower depending on the actual device breakdown voltage, with a modulation depth of typically 3 to 4 V at the second frequency. The waveform of the modulation, like that of the light source, is not limited to any particular form, but is typically sinusoidal. The output of the detector 4 is passed to a signal processor 24. An analogue-to-digital converter (ADC) (not shown) can be provided so that the analogue output signal of the single photon avalanche diode is converted to the digital domain and the signal processor 24 can employ digital signal processing (DSP). The signal processor may be present in the reader unit, so that the output from the single photon avalanche diode is typically transmitted from the detector head to the reader unit before further signal processing takes place. Alternatively, the signal processor may be located in the detector head.

The signal processor 24 can be implemented in dedicated electronic hardware, or in software running on a general purpose processor, or a combination of the two. In a preferred embodiment, a microprocessor 30 controls both the signal processor 24 that performs the analysis, and the signal generator 10. Thus the signal processor 24 has information on the light source modulation signal frequency and phase, and the detector bias voltage modulation frequency and phase.

The modulation of the bias voltage modulates the gain of the single photon avalanche diode detector 4. The light source 3, and hence the received fluorescence light are modulated at a first frequency, but the bias voltage of the single photon avalanche diode detector 4 is modulated at a second frequency, different from the first frequency. This enables a heterodyne measurement approach to be used by the signal processor 24 operating on an analysis signal at a frequency equal to the difference between the first frequency and the second frequency. Preferably the first and second frequencies differ by less than 10%, more preferably by less than 1%. The difference in frequency between first and second frequencies depends on the indicator system used but may be, for example 50 kHz.

According to another embodiment, the first and second frequencies can be nominally the same, but a varying phase shift is introduced between the signals (for example by delaying one signal with respect to the other, by a delay that continuously varies). As the phase shift changes each cycle, this is in fact the same as having two different frequencies. Preferably the introduced phase shift is swept rapidly.

From the signal being analysed, and knowing the frequency and phase of both the modulation of the light source 3 and of the modulation of the detector bias voltage, the signal processor 24 can determine the phase delay introduced into the system. The phase delay intrinsic to the sensor (which can be calculated either without any fluorophore present or with a sample of known fluorescence lifetime (known phase delay)) is deducted, providing a phase shift due purely to the fluorophore in the indicator system. This information can then be converted to a glucose concentration using appropriate calibration data. The required measurement result is then presented at output 26. The output measurement result can be displayed on a display (27 of FIG. 3) and/or can be logged in a memory (28 of FIG. 3) for later retrieval.

The above-described method essentially uses a single data point to derive the desired fluorescence-related information. However, according to a further preferred embodiment of the invention, a series of measurements are performed, but for each measurement a different phase shift and/or frequency difference is electronically introduced such that the phase angle can be controllably advanced or retarded. The two signal waveforms generated by the signal generator 10 are at the first and second frequencies that are different from each other, such that the relative phase of the signals at these frequencies will vary with time. However, the apparatus is in control, so that, for example, the waveforms at the two frequencies can be synchronised at a particular instant, and then the actual phase shift at any other time can be calculated. In one example, measurements are repeated with shifts in the frequency difference of 10 kHz, 20 kHz and 30 kHz. In addition a specific phase shift can be introduced at the point of synchronisation, so that the waveforms have a known initial phase difference. For each introduced phase angle shift, the modulation depth of the signal being analysed is obtained in order to effectively map out the phase-modulation space. The introduced phase angle may be incremented for example in steps of 5 degrees from zero to 180 degrees. The result is a series of data points that relate the modulation depths to the introduced phase angles. These data points constitute a graph that can be analysed e.g. by curve-fitting and/or comparison with calibration data of modulation depth relative to phase angle either with no sample present or with one or more standard calibration samples present. In general terms, results of measurements using different initial phase differences and/or different frequency differences can be aggregated, thus the overall measurement accuracy can be improved.

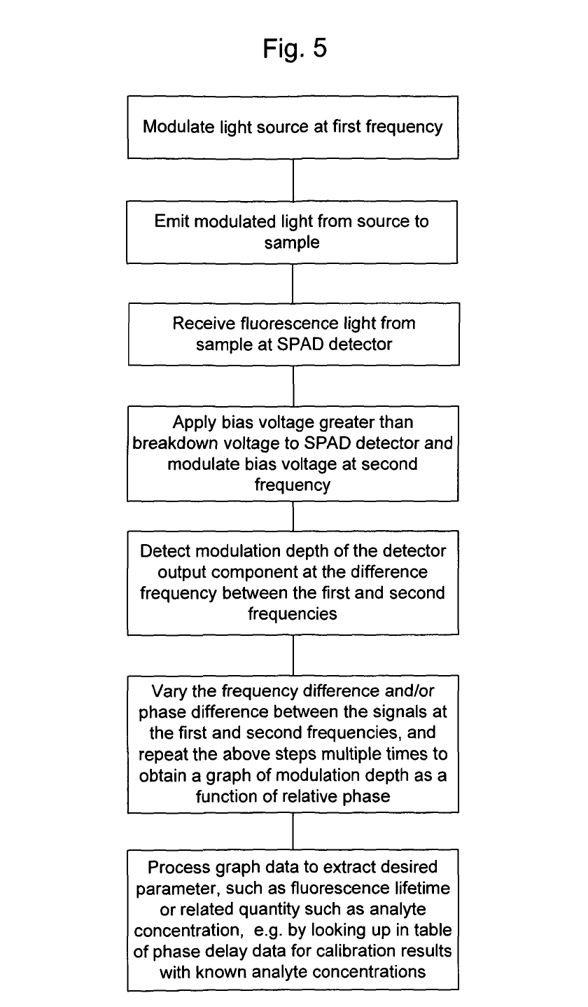

A summary of the method described above is depicted schematically in the flowchart of FIG. 5.

The whole sensor apparatus can be controlled by a microprocessor 30. Although FIG. 4 shows a number of discrete electronic circuit items, at least some of these may be integrated in a single integrated circuit, such as a field-programmable gate array (FPGA) or application-specific integrated circuit (ASIC).

Use of the sensor of the invention will typically involve attaching a disposable probe unit to the detector head and inserting the probe under the skin. The probe is typically inserted fully so that the lower surface of the detector head is in contact with the skin. Thus the tip of the probe is positioned approximately 3 to 7 mm under the skin. The sensor may be attached to the skin, for example using adhesive tape or by sutures to appropriate fixing points on the sensor. The reader unit is briefly connected to the detector head, for example for up to 30 seconds, preferably up to 20 seconds or up to 15 seconds. This period of time enables the measurement to be made and necessary data to be transferred to the reader unit.

In one embodiment of the invention, the detector head contains the light source, a power source and the detector, and the sensor is used to continuously monitor glucose levels. In this embodiment, since the detector head contains its own power supply, there is no need to provide connection between the reader and detector head prior to carrying out a measurement.

As used herein, continuous measurement of glucose concentration involves two or more, typically 10 or more, readings of the glucose concentration being taken automatically over a desired period, e.g. overnight. Thus, the microprocessor 30 is arranged for controlling the sensor apparatus so as to make a measurement of the glucose concentration automatically at defined intervals. This involves carrying out at least the steps of (b) providing incident light to the indicator system, (c) receiving fluorescent light emitted from the indicator system to generate an output signal, and (d) determining information related to the fluorescence lifetime of the fluorophore, two or more times at defined intervals. Typically, a measurement may be made once every 10 seconds to once every 10 minutes.

Typically, the output from the detector, optionally after suitable signal conversion, is transmitted wirelessly to the reader unit. Further signal processing may be carried out within the reader unit and the resulting data stored in memory capacity 28 and/or displayed using display 27. This embodiment enables data to be transmitted continuously to the reader unit, rather than on demand, and is particularly useful in the continuous monitoring of glucose levels overnight.

The invention has been described with reference to various specific embodiments and examples, but it should be understood that the invention is not limited to these embodiments and examples.

1. A glucose sensor for measurement of glucose in subcutaneous tissue, the sensor comprising:
a probe for subcutaneous insertion, the probe containing an indicator system comprising a receptor for selectively binding to glucose and a fluorophore associated with said receptor, wherein the fluorophore has a fluorescence lifetime of less than 100 ns;
a detector head which is optically connected to the probe and which is for location outside the body;
a light source; and
a detector arranged to receive fluorescent light emitted from the indicator system,
wherein the light source and detector are optionally located within the detector head;
wherein the sensor is arranged to measure glucose concentration in subcutaneous tissue by monitoring the fluorescence lifetime of the fluorophore.

2. A sensor according to claim 1, wherein the detector is a single photon avalanche diode.

3. A sensor according to claim 2, further comprising:
a driver arranged to modulate the light source intensity at a first frequency;
a bias voltage source arranged to apply a bias voltage to the single photon avalanche diode, wherein the bias voltage is modulated at a second frequency, different from the first frequency, and wherein the bias voltage is above the breakdown voltage of the single photon avalanche diode; and
a signal processor arranged to determine information related to a fluorescence lifetime of the fluorophore based on at least the output signal of the single photon avalanche diode.

4. A sensor according to claim 1 wherein the receptor is an enzyme or a compound containing one or more boronic acid groups.

5. A sensor according to claim 1, wherein the fluorophore has a fluorescence lifetime of 30 ns or less.

6. A sensor according to claim 1, wherein the fluorophore has a fluorescence lifetime of 20 ns or more.

7. A sensor according to claim 1, wherein the fluorophore is a non-metallic fluorophore.

8. A sensor according to claim 1, wherein the indicator system comprises a fluorophore-receptor construct which is bound to a hydrogel.

9. A sensor according to claim 8, wherein the hydrogel is a fluid hydrogel having a water content of at least 30% w/w.

10. A sensor according to claim 1, wherein the indicator system is provided as an aqueous solution.

11. A sensor according to claim 1, comprising (a) a non-disposable detector head and (b) a disposable probe unit comprising the probe and a connector arranged to connect the probe to the detector head.

12. A sensor according to claim 1, further comprising a reader unit arranged to connect to, or receive data from, the detector head, wherein the light source and detector are optionally located within the reader unit.

13. A sensor according to claim 12, wherein the detector head comprises the light source and detector and additionally comprises a power supply and a transmitter arranged to wirelessly transmit data relating to the output of the detector to a receiver, and wherein the reader unit comprises a receiver arranged to receive data transmitted by the transmitter.

14. A sensor according to claim 1, further comprising a microprocessor arranged for controlling the sensor to provide two or more measurements of glucose concentration at defined intervals and a memory arranged for storing information on the fluorescence lifetime data, or glucose concentration.

15. A disposable probe unit for use in a glucose sensor as defined in claim 1, comprising (a) a probe for subcutaneous insertion, the probe containing an indicator system as defined in any one of claims 1 or 4 to 10, and (b) a connector arranged to optically connect the probe to a detector head comprising, or being itself further optically connected to, a light source and a detector.

16. A detector head adapted for connection to a separate probe unit, wherein the detector head comprises a detector which is a single photon avalanche diode, the detector being arranged to receive light from the probe unit, the detector head being adapted to monitor fluorescence lifetimes of less than 100 ns.

17. A detector head according to claim 16, wherein the detector is adapted to monitor fluorescence lifetimes of 20 ns or more.

18. A method of measuring glucose concentration in subcutaneous tissue, which comprises
(a) inserting the probe of a sensor as defined in claim 1 into subcutaneous tissue;
(b) providing incident light to the indicator system from the light source;
(c) receiving fluorescent light, emitted from the indicator system in response to the light incident on the indicator system from the light source, using the detector and generating an output signal; and
(d) determining information related to the fluorescence lifetime of the fluorophore based on at least the output signal of the detector.

19. A method according to claim 18, which further comprises (e) wirelessly transmitting data relating to the output signal of the detector or to the fluorescence lifetime of the fluorophore, to a receiver located in a reader unit, wherein step (e) may be carried out either before or after step (d).

20. A method according to claim 18 wherein the detector is a single photon avalanche diode and the method further comprises the steps of:
(f) modulating the light source intensity at a first frequency; and
(g) applying a bias voltage to the single photon avalanche diode, wherein the bias voltage is modulated at a second frequency, different from the first frequency, and wherein the bias voltage is above the breakdown voltage of the single photon avalanche diode.

21. A method according to claim 18, wherein glucose concentration is monitored continuously by carrying out at least steps (b), (c) and (d) two or more times at defined intervals and storing the obtained information in a memory.
